(19) United States
(12) Patent Application Publication
(10) Pub. No.: US 2018/0108440 A1
(43) Pub. Date: Apr. 19, 2018

(54) SYSTEMS AND METHODS FOR MEDICAL DIAGNOSIS AND BIOMARKER IDENTIFICATION USING PHYSIOLOGICAL SENSORS AND MACHINE LEARNING

(71) Applicants: Jeffrey STEVENS, Santa Clara, CA (US); Sean CAFFEY, Santa Monica, CA (US); Nelson L. JUMBE, Mountain View, CA (US); Andrew URAZAKI, Duarte, CA (US)

(72) Inventors: Jeffrey STEVENS, Santa Clara, CA (US); Sean CAFFEY, Santa Monica, CA (US); Nelson L. JUMBE, Mountain View, CA (US); Andrew URAZAKI, Duarte, CA (US)

(21) Appl. No.: 15/786,190

(22) Filed: Oct. 17, 2017

Related U.S. Application Data
(60) Provisional application No. 62/409,042, filed on Oct. 17, 2016.
(60) Provisional application No. 62/429,906, filed on Dec. 5, 2016.

(51) Int. Cl. G16H 50/20; G06N 3/08; G06N 3/04; G10L 25/66; G10L 25/84; H04R 1/46

(52) U.S. Cl. CPC G16H 50/20; G06N 3/08; G06N 3/0445; H04R 1/46; G06N 3/0454; G10L 25/66; G10L 25/84; G06N 3/04

(57) ABSTRACT

Predictive healthcare systems utilize the signal produced by physiological and, in some embodiments, environmental sensors to infer, computationally, a physiological parameter of the patient. The physiological sensors may include a vibro-acoustic sensor in contact with a patient over at least the frequency band 0.001 Hz to 40 kHz and a bio-electric sensor. The physiological parameter may be the magnitude or existence of an internal process, such as blood flow; the presence of a biomarker; or the existence or likelihood of a disease. In some embodiments, the computational inference is based on additional data such as the patient's position and orientation and/or historical health information of the patient.

### CROSS-REFERENCE TO RELATED APPLICATION

This application claims priority to and the benefit of, and incorporates herein by reference in their entireties, U.S. Provisional Patent Application Nos. 62/409,042 and 62/429,906, which were filed on Oct. 17 and Dec. 5, 2016, respectively.

### FIELD OF THE INVENTION

In various embodiments, the present invention relates generally to monitoring of biological processes, and in particular to computationally inferring physiological conditions and their change over time from analysis of physiological and other data.

### BACKGROUND

### A. Acoustic Biosensing (Auscultation)

Stethoscopes are widely used by health professionals to aid in the detection of body sounds. The procedures for listening to and analyzing body sounds, called auscultation, are often difficult to learn due to the typically low sound volume produced by an acoustic stethoscope. Electronic stethoscopes have been developed to amplify the faint sounds from the body. However, such devices may suffer from distortion and ambient noise pickup. The distortion and noise are largely due to the performance of the acoustic-to-electrical transducers, which differ in operation from the mechanical diaphragms used in acoustic stethoscopes.

Traditional acoustic stethoscopes convert the movement of the stethoscope diaphragm into air pressure, which is directly transferred via tubing to the listener's ears. The listener therefore hears the direct vibration of the diaphragm via air tubes. Unfortunately, inefficient acoustic energy transfer via the air tubes causes diminished volume and sound clarity. Existing electrical stethoscope transducers are typically one of two types: (1) microphones mounted behind the stethoscope diaphragm, or (2) piezo-electric sensors mounted on, or physically connected to, the diaphragm.

Microphones mounted behind the stethoscope diaphragm pick up the sound pressure created by the stethoscope diaphragm, and convert it to electrical signals. The microphone itself has a diaphragm, and thus the acoustic transmission path comprises or consists of a stethoscope diaphragm, the air inside the stethoscope housing, and finally the microphone's diaphragm. The existence of two diaphragms, and the intervening air path, can result in excess ambient noise pickup by the microphone, as well as inefficient acoustic energy transfer. This inefficient acoustic energy transfer is a prevalent problem in the below-described electrical stethoscopes. Existing electronic stethoscopes use additional technologies to counteract this fundamentally inferior sensing technique, such as adaptive noise canceling and various mechanical isolation mountings for the microphone. However, these merely compensate for the inherent inadequacies of the acoustic-to-electrical transducers.

Piezo-electric sensors operate on a somewhat different principle than merely sensing diaphragm sound pressure. Piezo-electric sensors produce electrical energy by deformation of a crystal substance. In one case, the diaphragm motion deforms a piezoelectric sensor crystal mechanically coupled to the stethoscope diaphragm, resulting in an electrical signal. The problem with this sensor is that the conversion mechanism can produce signal distortion compared with sensing the pure motion of the diaphragm. The resulting sound is thus somewhat different in tone, and distorted compared with an acoustic stethoscope.

Capacitive acoustic sensors are in common use in high-performance microphones and hydrophones. A capacitive microphone utilizes the variable capacitance produced by a vibrating capacitive plate to perform acoustic-to-electrical conversion. A capacitive microphone placed behind a stethoscope diaphragm would suffer from the same ambient noise and energy transfer problems that occur with any other microphone mounted behind a stethoscope diaphragm.

Acoustic-to-electrical transducers operate on a capacitance-to-electrical conversion principle detecting diaphragm movement directly, converting the diaphragm movement to an electrical signal which is a measure of the diaphragm motion. Further amplification or processing of the electrical signal facilitates the production of an amplified sound with characteristics very closely resembling the acoustic stethoscope sound, but with increased amplification, while maintaining low distortion.

This is a significant improvement over the more indirect diaphragm sound sensing produced by the microphonic or piezoelectric approaches described above. Since the diaphragm motion is sensed directly, the sensor is less sensitive to outside noise, and the signal is a more accurate measure of the diaphragm movement. With an acoustic stethoscope, diaphragm movement produces the acoustic pressure waves sensed by the listener's ears. With an acoustic-to-electrical sensor, that same diaphragm movement produces the electrical signal in a direct manner. The signal is used to drive an acoustic output transducer such as earphones or headphones, to set up the same acoustic pressure waves impinging on the listener's ears.

While acoustic-to-electrical transducers overcome many of the inherent problems faced by earlier stethoscope designs, it adds considerable white noise to the signal. White noise is a sound that contains every frequency within the range of human hearing (generally from 20 hertz to 20 kHz) in equal amounts. Most people perceive this sound as having more high-frequency content than low, but this is not the case. This perception occurs because each successive octave has twice as many frequencies as the one preceding it. For example, from 100 Hz to 200 Hz, there are one hundred discrete frequencies. In the next octave (from 200 Hz to 400 Hz), there are two hundred frequencies.

As a result, the listener has difficulty discerning the human body sound from the white noise. For sounds of the body with higher intensities (i.e., louder sounds) the listener can hear the body sounds well, but lower-intensity sounds disappear into the background white noise.

FIG. 1 shows the frequency bands associated with various bodily sounds of clinical interest. The figure reveals that most of the significant cardiac, respiratory, digestive, and movement-related sound information occurs in frequencies below those associated with speech, and in fact most information lies below the threshold of human audibility (since this increases sharply as frequency falls below about 500 Hz. Noises caused by movements of muscles, tendons, ligaments, adjacent organs in the chest cavity, etc. are rarely detected and analyzed today due to their low frequency band and the limits of conventional detection approaches. Hence, improved detection techniques would facilitate acquisition of acoustic signals that, alone or in combination with other biologically relevant signals and information, could be used to monitor physiological conditions and diagnose disease.

### B. Electrical Biosensing

The dipole is the elemental unit of cardiac activity. Each dipole consists of a positive (+) and negative (−) charge generated by the action of ion channels. As activation spreads, the sources sum together and act as a continuous layer of sources. Stated simply, an electric dipole consists of two particles with charges equal in magnitude and opposite in sign separated by a short distance. In the heart, the charged particles are ions such as sodium (Na⁻), potassium (K⁺), calcium (Ca²⁺), phosphates (PO₄³⁻), and proteins. The separation is the distance across the cardiac cell membrane. Because they are too large to pass through the small cell membrane channels, the negatively charged particles remain in the cell, whereas the positive ions move back and forth through specific channels and “ion pumps” to create polarization and depolarization across the membrane.

If enough dipoles are present together, they create a measurable voltage. Resting cardiac cells within the heart are normally at −70 mV. This means that at rest, there is naturally a charge imbalance present in the heart. This imbalance, called polarization of the cell, attracts positive ions toward the interior of the cell. When a cardiac cell is activated by an outside stimulus, channels in the cell membrane activate, and the excess positive ions outside of the cell rush into the cell. This process, called depolarization, makes the cell less negatively charged and is associated with “activation” of the cardiac cell. When millions of these cells activate together, the heart contracts and pumps blood to the rest of the body. The combined activation of these cells generates enough voltage to be measured on the surface of the skin by an electrocardiogram (ECG). The resulting intracardiac electrogram (EGM) extends beyond the area of the dipole signal by a factor of five, reducing resolution and acuity.

For over 100 years, voltage has been the major electrical measurement in cardiac medicine. Voltage readings, however, include both the localized charge (dipole density) as well as the sum of the surrounding sources, providing a broad, blended view of cardiac activity that limits diagnostic resolution.

### SUMMARY

Embodiments of the present invention utilize the signal produced by physiological and, in some embodiments, environmental sensors to infer, computationally, a physiological parameter of the patient. The physiological sensors, mostly passive sensors in all embodiments, may include a vibro-acoustic sensor in contact with a patient over at least the frequency band 0.001 Hz to 40 kHz and a bio-electric sensor to measure electrical fields and electrical impulses, and various other sensors described in detail herein. The physiological parameter may be the magnitude or existence of an internal process, such as blood flow; the presence of a biomarker; or the existence or likelihood of a disease. In some embodiments, the computational inference is based on additional data such as the patient's position, orientation, environmental, and/or historical health information of the patient. Biosensors in accordance herewith separate dipole density from voltage to increase diagnostic specificity and capability.

Accordingly, in one aspect, the invention pertains to a system for receiving and transducing biological events into electrical signals and diagnosing a medical condition based thereon. In various embodiments, the system comprises a sensor array comprising a vibro-acoustic sensor for measuring body sounds of a patient and a bio-electric sensor for measuring a bio-electric signal of the patient; a processor; and a machine learning module, executable by the processor and trained on signals characteristic of the sensor array, the machine learning module receiving signals from the sensors and, based on the training, outputting a probability indicative of a physiological condition. For example, the physiological condition may be a biomarker as defined below.

In some embodiments, the sensor array further comprises one or more sensors for measuring at least one environmental stimulus or condition. For example, the environmental stimulus or condition may be at least one of skin temperature, ambient temperature, barometric pressure, 9-axis motion, geolocation, location-dependent real-time weather conditions, galvanic skin response, or pollution.

Alternatively or in addition, the sensor array comprises at least one sensor for measuring at least one of wavelength transmittance/absorbance, oxygen saturation, ambient temperature, skin temperature, body core temperature, ACG, BCG, ECG, EMG, EOG, EEG, UWB, VOC excretion or vocal tonal inflection. The system may also include one or more optical sensors.

In various embodiments, the system further comprises a database of longitudinal health records, the health record of a patient being monitored by the sensors providing an input to the machine learning module. The machine learning module may be one or more neural networks, e.g., a recurrent neural network, a feedforward neural network, or an ensemble of neural networks. The machine learning module may be local or remote from the sensors and in communication therewith via a network.

The vibro-acoustic sensor and the bio-electric sensor may each produce time-varying signals in a time-synchronized fashion. For example, the signals may be received by the machine learning module as catenated raw amplitude sequences or as combined short-time Fourier transform spectra.

The sensor array may be connected by wires or may communicate wirelessly, e.g., for purposes of telemetry, control, and/or power transference. The system may also include at least one acoustic stimulus generator.

As used herein, the terms “approximately,” “roughly,” and “substantially” mean ±10%, and in some embodiments, ±5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1 shows the frequency bands associated with various bodily sounds of clinical interest.

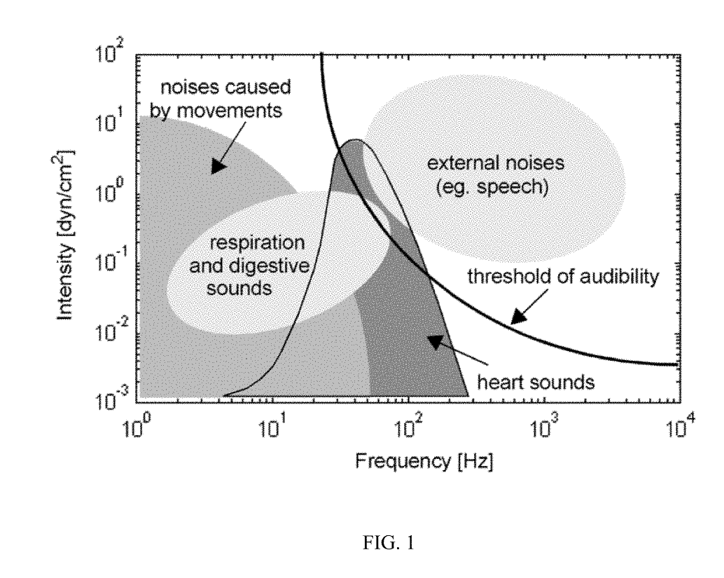

FIG. 2 schematically illustrates a representative architecture implementing the functionality of the present invention.

### DETAILED DESCRIPTION

### A. Core Architecture

Embodiments of the present invention pertain to wearable sensor arrays that can identify and monitor diagnostic digital biomarkers (as defined below). Various embodiments feature advantageous improvements to sensor sensitivity, specifically vibro-acoustic and bio-electric sensors that can monitor sounds, vibrations and electrical fields and impulses of the target living organism (e.g., a human patient), then apply techniques of machine learning to monitor and diagnose healthy vs. disease states. Machine learning may be either supervised learning (parametric/non-parametric algorithms, support vector machines, kernels, neural networks), unsupervised learning (clustering, dimensionality reduction, recommender systems, deep learning), or combinations thereof. Embodiments of the invention may utilize one or more physiological sensors as well as environmental sensors and other sources of health-related information.

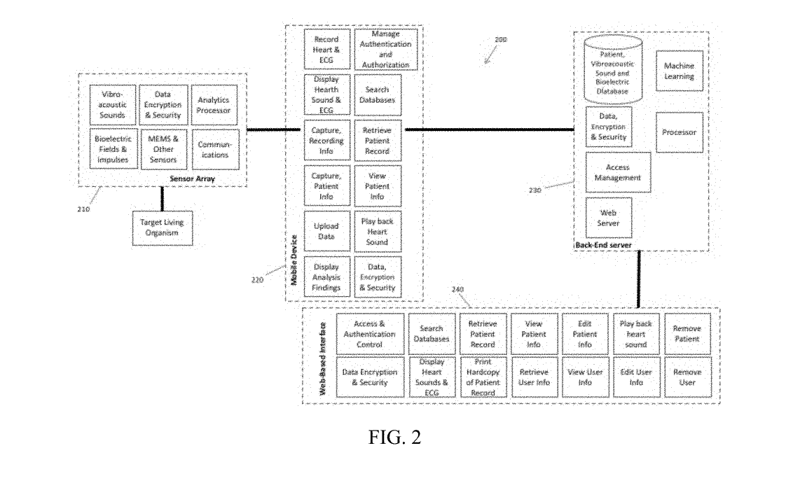

Refer first to FIG. 2, which illustrates a system-level view of a representative topology 200 implementing an embodiment of the present invention, which includes various optional components. The system 200 includes one or more sensors 210; an optional mobile device 220 that receives and controls sensor signals and relays them to a back-end server 230, and also receives processed data from server 230 for display to the user; and a web-based interface 240, which may exist separately from or serve as an alternative to mobile device 220 with additional capabilities including access to relevant patient information. Typically, the interface 240 is implemented on a general-purpose computer or workstation, while the mobile device may be a “smart” phone or tablet running an on-board application (“app”). In general operation, one or more sensors 210 detect one or more patient conditions and output electrical (analog and/or digital) signals indicative of the sensed condition. Sensors may be located individually, in redundant clusters (e.g. one cluster containing one vibro-acoustic sensor and two non-contact bio-electric field/impulse sensors). Sensors may also be located internal to the patch 210a or external to the patch 210b (interfacing the body or environment). Suppose, for example, that the sensors monitor cardiac parameters and that the system is configured to predict whether the patient will go into cardiac arrest during a medical procedure. In this implementation, the sensor array 210 may include a vibro-acoustic sensor, a plurality of bio-electric sensors for an ECG unit, and a MEMS (or other) sensor to detect the patient's position and/or orientation; the outputs of all of these sensors are relevant to the likelihood of cardiac arrest, and are provided to a machine learning module in the server 230. As described in greater detail below, the machine learning module predicts the likelihood of cardiac arrest given the incoming signals from the sensors 210. For example, the signals may be repeatedly sampled over a time window and the synchronized raw signal amplitude patterns from each sensor catenated into a single feature vector that is used to query the machine learning module, which has previously been trained on similar feature vectors. The raw data may be stored in a time-indexed log in a memory to facilitate synchronization, and may also be stored in a database to facilitate selective retrieval by the mobile device 220 or interface 240. For example, successive one-second windows of data may be provided to the machine learning module, which each time returns a likelihood of cardiac arrest. More generally, the database may be used to store biomarkers based on data obtained across multiple patients. For example, the data gathered from patients' chests prior to and during heart attacks can be used to create novel digital biomarkers for diagnosing and predicting cardiac arrest. Specific subset data of the digital biomarker may further be used for predicting future tangential or causative diseases.

The processing rate of the machine learning module limits the rate at which the one-second data windows can be ingested and processed—e.g., if the machine learning module needs three seconds to process data and return a result, the throughput rate is ⅓ sec⁻¹, and analysis findings are displayed on the device 220 (“Display Analysis Findings”) and updated every three seconds. The manner in which these findings are displayed depends on design preferences; a raw likelihood may be displayed in percentage terms, or a color code (e.g., red, yellow and green graphics) indicative of the current risk level may be displayed instead or in addition to the percentage. Device 220 may receive raw probability data from server 230 and format a display using on-board software, or may receive a displayable image in markup format from a conventional web server module in server 230; the received image is displayed by device 220 in a browser app.

In addition, the clinician may wish to view the sensor data directly. To support this, the device 220 may include mass storage for caching a time window of sensor data (“Record Heart & ECG”) and displaying the data in a useful format. As used herein, the term “display” is not limited to a visual rendering on a screen but also includes aural reproduction, e.g., of a sensed heartbeat, or tactile reproduction as discussed, for example, in U.S. Ser. No. 15/471,815, filed on Mar. 28, 2017 and entitled “Haptic Feedback And Interface Systems,” the entire disclosure of which is hereby incorporated by reference. Using mobile device 220, the user may query the server database for earlier records (e.g., ECG traces) for comparative purposes, and may request patient records. The queries of sensor raw data and the physician's understanding and interpretation of such data may also serve as input to the machine learning module. To support privacy and security requirements, the devices 220, 240 may include data encryption and authentication software that serves as a front end to an electronic medical records (EMR) facility.

The sensor array 210, server 230, and mobile device 220 and/or interface device 240 may communicate via one or more networks. The term “network” is herein used broadly to connote wired or wireless networks of computers or telecommunications devices (such as wired or wireless telephones, tablets, etc.). For example, a computer network may be a personal area network (PAN), a local area network (LAN) or a wide area network (WAN). When used in a PAN networking environment, computers and sensor arrays may be connected to the PAN through radios such as Bluetooth. When used in a LAN networking environment, computers may be connected to the LAN through a modem, network interface or adapter. When used in a WAN networking environment, computers typically include a modem or other communication mechanism. Modems may be internal or external. Networked computers may be connected over the Internet or any other system that provides communications. Some suitable communications protocols include TCP/IP, UDP, and Bluetooth. For wireless communications, protocols may include IEEE 802.11x (“Wi-Fi”), Bluetooth, ZigBee, IrDa, near-field communication (NFC), or other suitable protocol. Furthermore, components of the system may communicate through a combination of wired or wireless paths, and communication may involve both computer and telecommunications networks.

It should also be stressed that the distribution of functionality illustrated in FIG. 2 is representative only. The functionality may be spread arbitrarily over multiple intercommunicating devices, or may be centralized in a single device, e.g., a laptop or even a tablet with sufficient processing capacity. To support privacy and security requirements, the functionality may also be spread over multiple devices by taking into consideration data encryption and authentication requirements of EMR. Additionally, functionality may be spread over multiple devices based on disposability and reusability (e.g., sensor arrays may be disposable, whereas the processing, data storage and communication modules may be reusable).

The system 200 (or server 230) may be or include a general-purpose computing device in the form of a computer including a processing unit, a system memory, and a system bus that couples various system components including the system memory to the processing unit. Computers typically include a variety of computer-readable media that can form part of the system memory and be read by the processing unit. By way of example, and not limitation, computer-readable media may comprise computer storage media and communication media. The system memory may include computer storage media in the form of volatile and/or nonvolatile memory such as read only memory (ROM) and random access memory (RAM). A basic input/output system (BIOS), containing the basic routines that help to transfer information between elements, such as during start-up, is typically stored in ROM. RAM typically contains data and/or program modules that are immediately accessible to and/or presently being operated on by processing unit. The data or program modules may include an operating system, application programs, other program modules, and program data. The operating system may be or include a variety of operating systems such as Microsoft WINDOWS operating system, the Unix operating system, the Linux operating system, Apple OS X, or another operating system or platform.

The computing environment may also include other removable/non-removable, volatile/nonvolatile computer storage media. For example, a hard disk drive may read from or write to non-removable, nonvolatile magnetic disks. A magnetic disk drive may read from or writes to a removable, nonvolatile magnetic disk, and an optical disk drive may read from or write to a removable, nonvolatile optical disk such as a CD-ROM, DVD-ROM, Blu-ray, or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The storage media are typically connected to the system bus through a removable or non-removable I/O interface.

The processing unit that executes commands and instructions may be a general purpose computer, but may utilize any of a wide variety of other technologies including a special-purpose computer, a microcomputer, mini-computer, mainframe computer, programmed microprocessor, microcontroller, peripheral integrated circuit element, a CSIC (customer-specific integrated circuit), ASIC (application-specific integrated circuit), a logic circuit, a digital signal processor, a programmable logic device such as an FPGA (field-programmable gate array), PLD (programmable logic device), PLA (programmable logic array), precise timing protocol component (PTP) providing a system with a notion of global time on a network, RFID processor, smart chip, or any other device or arrangement of devices that is capable of implementing the steps of the processes of the invention.

The various modules shown in FIG. 2, including the machine learning module, may be implemented by computer-executable instructions, such as program modules, and executed by a computer. Generally, program modules include routines, programs, objects, components, data structures, etc. that performs particular tasks or implement particular abstract data types. Any suitable programming language may be used in accordance with the various embodiments of the invention. Illustratively, the programming language used may include assembly language, Accord, Apache Mahout, Basic, C, C++, C*, Caffe, Clojure, Cloudera Oryx, COBOL, ConvNetJS, Cuda, PyTorch, Theano and TensorFlow, dBase, DeepLearn.js, Forth, FORTRAN, GoLearn, Haskell, H20, Java, Mathematica, MATLAB, Modula-2, Pascal, Prolog, Python, R, REXX, Scala, and/or JavaScript, Scikit-learn, Shogun, Spark MLlib, Weka for example. Further, it is not necessary that a single type of instruction or programming language be utilized in conjunction with the operation of the system and method of the invention. Rather, any number of different programming languages may be utilized as is necessary or desirable.

While computer system 200 is described herein with reference to particular blocks, it is to be understood that the blocks are defined for convenience of description and are not intended to imply a particular physical arrangement of component parts. Further, the blocks need not correspond to physically distinct components. To the extent that physically distinct components are used, connections between components (e.g., for data communication) can be wired and/or wireless as desired.

Having described the general features of the system 200, the sensor array and machine learning module will now be described in greater detail.

### B. Sensors

B.1 Vibro-Acoustic Sensors

The sensor array 210 desirably includes a vibro-acoustic transducer arrangement optimized for sensing and transducing acoustic phenomena occurring within a target living organism's or patient's body, and manifesting themselves at the skin surface with frequencies ranging from 0.001 Hz to 40 kHz. Strategies for effectively coupling to the skin include judicious mismatching of mechanical impedance, the use of impedance-matching gels or liquids, a shaped (e.g., domed) pickup, material selection, and/or a peripheral leaf-spring arrangement permitting relative movement between inner and peripheral diaphragm portions as described, for example, in U.S. Ser. No. 15/471,812, filed on Mar. 28, 2017 and entitled “Vibro-Acoustic Transducer,” the entire disclosure of which is hereby incorporated by reference.

In various embodiments described in the '812 application, the sensor device comprises a diaphragm having an outer peripheral portion and an inner portion. The inner movable portion is attached to the outer portion by a plurality of leaf springs constraining relative movement between the inner portion and the peripheral portion. The sensor device also includes a coil disposed over at least one side of the diaphragm, and at least one magnet operatively disposed with respect to the coil to cause current to flow through the coil upon relative movement between the movable portion and the peripheral portion. The spring stiffness or spring compliance of the leaf springs may be selectively chosen to optimize the frequency response of the sensor.

In some embodiments described in the '812 application, the inner portion is fixed and the outer peripheral portion is movable with respect thereto; in other embodiments, the outer portion is fixed and the inner peripheral portion is movable with respect thereto. For example, in a particular embodiment, the outer fixed portion of the diaphragm has a shape and the inner movable portion is defined within a plurality of slots through the diaphragm and arranged in a series. The series defines a closed sequence concentric with and having the shape of the outer fixed portion, and each pair of slots is parallel and has an overlap portion and a non-overlap portion, the overlap portion defining an intervening strip corresponding to one of the leaf springs. In some cases, the slots are filled with a thixotropic material. In some embodiments, the coil and the at least one magnet are circular, while in other embodiments, one or both have a different shape.

More generally, the vibro-acoustic sensor used herein may be optimized to the viscoelastic properties of target tissues in order to maximize the quality of data gathered. Optimization factors include but are not limited to the viscoelastic parameter range of the target tissue, target living organism specific or patient specific variations in tissue composition, and sensor-attachment interface material. Target tissue viscoelastic parameters can be characterized broadly (e.g., the whole chest cavity) or restricted to localized areas or target tissue response (e.g. cardiac functionality, factoring out pulmonary input factors). For example, it is known that individual target tissues have specific viscoelastic factors that contribute to the desired target vibro-acoustic information to be detected—e.g., for a cardiac target, the major factors are the muscular contractions and blood flow. Furthermore, measuring a pregnant woman's abdomen creates additional challenges for the measurement or propagation of soundwaves, vibrations, light or electromagnetic waves due to the complex interface of new tissue and water layers caused by the presence of the amniotic cavity, uterine wall and other collagenic and tissue interfaces not normally found in adults. These tissue interfaces, which grow and move with fetal maturation and movement, can change the propagation of sound, vibrations and light, making it more difficult to record inputs or image inside the body.

Additionally, the correlations between the mechanical properties and material properties of certain muscular tissues may be monitored in real time to characterize their viscoelastic properties. Such information is used to generate stress and strain models, characterize the creep and strain-rate sensitivity of biological tissues (e.g., skeletal musculature atrophy and bone porosity), and monitor environmental and disease effects on tissue over periods of time (e.g., changes in bone viscoelasticity over time in microgravity and zero-gravity conditions).

Sensor attachment interface material may additionally affect the quality of the obtained vibro-acoustic signals. The fabric, gel patch, adhesive, or other interface is selected for optimal vibro-acoustic damping. Furthermore, the center and peripheral edges of the sensor may comprise or consist of differing material or differing amounts of material to further control viscoelastic damping.

B.2 Non-Contact Bio-Electric Sensors

The sensor array 210 may include sensors for one or more bio-electric time-varying signals, i.e., the change in electric current produced by electrical potential differences across a specialized tissue, organ or cell system like the nervous system.

A bio-electric sensor may be capacitive so it does not rely on ohmic contact to the body for measuring bio-electrical signals (see, e.g., U.S. Pat. Nos. 3,882,846 and 3,500,823, the contents of which are incorporated herein by reference). This facilitates data collection across the target living organism (e.g. human body), and confers the ability to measure electrocardiography and other electrical fields and impulses without direct skin contact. Measurements such as ECG depend on being able to extract the small electrophysiological signals from the much larger noise signals. Unlike the silver/silver chloride (Ag/AgCl) electrodes used in clinical settings, bio-electric sensors in accordance herewith may make a high-impedance contact to the skin. This allows accurate and convenient measurement of the ECG. For such sensors, no gel, paste or other preparation is required at the sensor-skin interface. The connection is not affected by changes in skin impedance brought on by perspiration.

Data from sensor arrays as described herein may include near real-time, ambulatory electrocardigraphy (ECG), vectorcardiography (VCG), ballistocardiography (BCG), phonocardiography (PCG), and acoustic cardiography (ACG). ACG synchronizes cardiac sounds with the bio-electric sensor's electrocardiogram information and provides a comprehensive assessment of both mechanical and electrical functions of the heart. ACG is applied to heart failure diagnosis and ischemic heart disease detection, as well as other diseases including LV hypertrophy, pericarditis, sleep apnea and ventricular fibrillation. BCG measures cardiac ballistic forces with ultra-high resolution, enabling blood pressure to be measured “beat-to-beat” non-invasively with a wearable sensor. Vector cardiography, the electrical depolarization of the human heart, can be estimated and, if desired, visualized using vibro-acoustic data generated using sensors described herein.

The sensor data may be used to extrapolate various models and used to diagnose many heart diseases in just a few beats. Furthermore, sensor arrays in accordance herewith may contain memory and processing in a lightweight package and can easily transmit data wirelessly or via a wired connection. Various embodiments may additionally be indicated for heart failure follow-up in homes, clinics and hospitals as well as in the microgravity or zero-gravity of space.

Additionally or alternatively, in certain embodiments, specific target tissues may be locally stimulated to produce a response to be recorded by the sensor array (e.g., acoustic signals introduced into the body from a speaker can actively change the data captured by the above mentioned vibro-acoustic sensor) or a response from the target living organism (e.g. fetus). The stimulation mechanisms may be sound applied to the skin, vibrations, ultrasound, photonic, laser, a set period of motion (wave), or other bands within the electromagnetic spectrum, etc. This functionally can be used to stimulate certain conditions such as stress, functional movement, and various other activities.

B.3 3D and 4D Imaging

Certain embodiments of the system have advantageous qualities for imaging by monitoring the vibro-acoustic and bio-electric signals coming from certain tissues. While conventional imaging systems may operate by inducing a sound and then interpreting the reflection, embodiments of the present invention performs the inverse whereby the signal source is coming towards the sensors without the need for a reflection. For example, sonar, radar and ultrasound transmit an electromagnetic signal and then interpret the reflection off the object (e.g., different tissues, amnion, organs, abscess, other localized infections, etc.) being studied. Sensors in accordance herewith, when placed in multiple locations on the body, may directly record the vibro-acoustic and bio-electric signals to create a 3D map of the signals and construct an image utilizing the collected signals. The system works in a fashion similar to the hammerhead shark, which utilizes bio-electric sensors to visualize the location and approximate size of prey buried under the sand before attacking. In much the same way, embodiments of the present invention measure the amplitude and voltage potential directly across the contours of the patient's body as well as sounds, vibrations and pressure waves through a networked array of bio-electric, vibro-acoustic sensors and optionally including other sensors mentioned herein (e.g., position, temperature, UWB, etc.) while incorporating environment sensors as well). In some embodiments of the invention, a finite-element model mesh is used to approximate the cardiac geometry from 1) time-gated, reality-based structural information, 2) continuous target tissue pressure, and/or 3) tissue elastance determined from bio-electric and vibro-acoustic data. Rendered tissue or fetal volumes may be shown in 3D as well as displayed in time-resolved 4D animations.

This imaging approach can be used to image the fetal womb. The networked bio-electric and vibro-acoustic and other sensors (such as for position, to observe changes in fetal structures and tissues when the mother is supine or prone, for example) measure bio-electric signals and sounds, vibrations and pressure waves coming from the fetal heart, circulation and other functional areas of the fetal and maternal body to turn in these signals into images and data for machine learning. Furthermore, interference of the signals from the fetus will be disrupted by tissues external to the fetus (such as the amniotic cavity, amniotic fluid volume, compliance of the uterine wall, or blood flow exchange across the placenta, for example) which can inform on dimensions, compliance, stiffness (such as a digital palpation) using the sensors surrounding the womb. These measurements and imaging can either be recognized instantly through pattern recognition using machine learning or in some cases the pattern can change over time to better observe and identify diseased or “healthy” states, providing reassurance (so no action or intervention needs to occur in an otherwise confusing situation possibly requiring premature cesarean section or other potentially dangerous intervention) or indicating the need for clinicians to escalate treatment and/or intervene.

In one embodiment, the vibro-acoustic sensor, bio-electric sensors and other sensors mentioned herein are woven into a flexible garment placed around the entire womb of the expectant mother. The system then records vibro-acoustic signals from the moving fetus's heart, blood flow turbulence, motion, and other biological sounds. Furthermore, the “signature” of the fetus's bio-electric signals may reveal variations in mass, position, and state of the fetus and overall heath or disease. By measuring both vibro-acoustic and bio-electric fields either instantaneously or over time, embodiments of the invention may search for patterns of healthy vs. disease states, which may be correlated with environmental information (growth chart from medical record, weight of mother, etc.) and physiology scores (i.e., heart rate variability, fetal kicks per unit time, etc.) in order to study thousands of babies and their different biomarkers (e.g., Gestational Diabetes Mellitus, preeclampsia, early delivery, cesarean birth, having a big baby which can complicate delivery, infection, etc. or predicting a baby born with having low blood sugar, breathing problems, jaundice, cord strangulation, hypoxia, etc.). In another embodiment, ultrasound or UWB waves can be used as an adjunct to the passive system above in order to potentially improve the resolution of features, compliance of tissues, or more accurate changes.

This approach can also be used on sound waves emanating from inside the body to assess the potential riskiness of atherosclerotic plaques, compliance of arteries and arterioles along the heart or elsewhere, cardiac output, cardiac enlargement, carotid intimal medial thickness, to screening for chronic liver or kidney disease (an acoustic palpation is able to determine the stiffness or compliance of the liver or kidneys), or to improve drug delivery by localizing the effects: bio-electric signatures change based on metabolic activity and increased or decreased emittance of electric impulses, and so can reveal the effects or effects of pharmaceutical products over time, and therefore combine with other lifestyle and health information collected from the sensors associated with a particular patient. This virtual palpation technique images tissue stiffness differences associated with different pathologies. Systems in accordance herewith can be used as an adjunct to conventional ultrasound for clinicians, since images acquired using the vibro-acoustic sensor in the range of 10 kHz to 40 kHz can be compared to conventional ultrasound images to provide additional information and, often, improved contrast.

One specific response outcome obtained by applying stimuli is acoustic- and bio-electric-based 3D imaging of various tissues throughout the body. As mentioned above, the vibro-acoustic sensor data and bio-electric sensor data can be obtained and display three dimensional images of the internal structure of the target living organism. Compared to conventional imaging methods through which data is obtained using high-powered energy sources (e.g., X-ray, ultrasound, gamma rays, etc.), this low-powered alternative can be realized as a wearable to generate real-time and time-lapsed 3D imaging in a manner that is completely passive, low cost and safe (even ultrasound imaging can cause cavitation of tissues, which may not be safe when applied to fetuses or across sensitive areas of the body).

In various embodiments, other physiological sensors including, but not limited, to a pulse oximeter for wavelength transmittance/absorbance and oxygen saturation, an ambient skin/core temperature thermometer, optical sensors, camera systems, photonic sensors, infrared sensors, near- and far-infrared sensors, and a UV sensors for overall physical assessment, ultrasound for internal organ scan, electromyography (EMG) for mechanical properties of muscles at rest and in contraction, electroencephalogram (EEG) for electrical activity for functional status of the brain, electrooculography (EOG) for changes in resting/active electric potentials of the eye retina function, and/or a volatile organic compound (VOC) detector for organic compounds in excretions (e.g., perspiration and breath) may be employed. Such sensors may be placed in separate, non-physically tethered arrays (e.g., one array for an EEG may be in the form of a cap, one array for a VOC may be in the form of a patch so that perspiration from a target region can be tested). Some or all of these other physiological sensor outputs may be relevant for evaluation of the cardiopulmonary state in this example. In certain embodiments, additional physical sensors are incorporated into the sensor array. Another exemplary physiological and imaging sensor is the ultra-wideband (UWB) sensor which is a low power, non-ionizing electromagnetic wave, high-penetration alternative to other imaging methods (MRI, X-ray), making it suitable for a wearable or implantable application.

In one embodiment, the optical sensor is a pulse plethysmograph (PPG) used to measure one or more of various conditions including heart rate, blood oxygen saturation, body hydration, severity of venous reflex disease, venous function, and cold sensitivity.

In various other embodiments, other biosensors can be used to obtain data through specific biorecognition of various elements (e.g., enzymes, antibodies, protein, nucleic acid, ion receptors, cell types) in samples obtained from the target living organism. Specific biosensors include but are not limited to surface plasmon resonance (SPR) biosensors for detecting proteins and toxins, evanescent wave fluorescence biosensors for detecting biodefence and toxins, bioluminescent optical fiber biosensors for detecting genotoxins, waveguide interferometric biosensors for detecting cellular response and viruses, ellipsometric biosensors for detecting viral receptors, reflectometric interference spectroscopy biosensors for detecting xenobiotics and tumor cells, and surface-enhanced Raman scattering biosensors for detecting cancer proteins.

B.3 Environmental and Other Sensors

Sensor array 210 may include one or more microphones. For example, tonal inflection changes can reveal mood changes or emotional response, which may then be correlated to the simultaneously measured physiological response. Tonal response may further show a change in psychological disposition.

In certain embodiments, one or more environmental sensors are incorporated into the sensor array. Environmental sensors can measure skin temperature, ambient temperature, barometric pressure, 9-axis motion detection (3-axis magnetometer, 3-axis accelerometer, 3-axis gyroscope), which may be realized in MEMS form), geolocation, location-dependent real-time weather conditions (wind, humidity, rain, specific storm conditions, UV index), galvanic skin response, and pollution (air, light, noise, water, soil, proximal radioactivity, visual and other ambient conditions and contaminants). A sensor (or sensor system) may be used to track the patient's position and/or orientation, since these may be relevant to a biomarker. The patient's location can be improved by Wi-Fi, Bluetooth, and integration of various wireless communication protocols for more accurate location determination. Within a “smart home” (with connected devices as described above), systems in accordance herewith may be connected to “Internet of things” devices whose states can inform on the health status of a patient and whose operation may enhance patient convenience. Home sensors, for example, can include access to medicine containers (smart containers that show when medicine was administered, such as when bottle was opened and closed) and smart toilets (reading urine, fecal, or other metabolite analysis). Clothing cameras may be used to determine what the patient is wearing; over time, the patient's clothing habits can inform on the overall change in a patient's mental state (such as a depressive, euphoric or stressed emotional state). Smart scales will inform on weight which can give insight into a patient's hydration status, and when combined with other sensor readings may provide data on the daily routine and habits that may correlate to specific outcomes.

B.4 Biomarker Identification and Use

As used herein, the term “biomarker” refers to an association between one or more measurable signals and one or more physiological or disease states. These signals are measured using the sensors 210, and analysis thereof using machine learning techniques, as described below, can be used to detect the presence and state of a biomarker in a patient. For example, a biomarker may be expressed in terms of a probability estimated using linear regression or a neural network applied to input signals from one or more sensors.

For example, with enough population data from one or more (and desirably many) demographics, a normal standard of individuals who have not manifested precursor symptoms or symptoms of known disease states may be created and specific deviations therefrom can be assigned as separately diagnosable disease states (e.g., type of disease, precursor event identification, progression status, treatment options and recommendations, etc.). While no individual is “healthy” s/he may be at a baseline current state where certain disease states are either undetectable, misdiagnosed, or have yet to manifest currently detectable symptoms. With accumulation of population data, a better understanding of “health” can be contextualized and monitored on a spectrum of higher precision. As a result, any disease state and/or associated precursors can be monitored for progression and regression including first and second derivatives to obtain safety and efficacy data of treatments (e.g. pharmaceutical therapy, physical therapy, cognitive therapy, spiritual therapy, etc.) The result is a database of “virtual patients” for evaluation of new interventions by “phenotypes,” enabling eventual customization of treatment by patient characteristics. In addition to direct/absolute measures, derived measures including heart rate variability, FFT, pulse transit time, harmonic expansion/compression, spectrograph amplitude/frequency envelope, etc. may be better predictors of specific biomarkers. For each different population (e.g., a population in microgravity, zero gravity, altered acceleration/simulated gravity), the standard digital biomarker may be adaptively calibrated as certain disease states may have different contributing factors, attributes, progression rates, and treatments. For example, in microgravity environments, the heart does not work as hard due to the lowered resistance of gravity, thereby causing the heart to become approximately 10% more spherical in the micro-gravity of low Earth orbit and zero gravity of outer space. Changes in relevant digital biomarkers of astronauts from normal gravity to microgravity to zero gravity environments may be observed using the techniques and systems described herein.

Diagnostic digital biomarkers may be tailored for each individual patient by including as input a patient's longitudinal health records as well as recorded or self-reported family history. Once the individual's personal information is integrated, a personalized digital biomarker or phenotypic fingerprint is generated, thereby allowing for the possibility of customized healthcare. Such information may be further strengthened by correlations found in genotypic similarities through DNA banks. Additional sources of data and types of information of interest include but is not limited to: (a) disease data of the more than 30,000 diseases currently known in medical fields (e.g. cardiovascular, nervous system, inflammation, immune, metabolic, infectious disease, etc. and various combinations thereof) and/or (b) microbiome, transcriptome, proteome, metabolome, etc. to further understand gene expression. The above information is currently and will further be accumulated in databases. It is well known that certain genetic subsets of the population suffer from increased hypertension and increase response to sodium, and with this type of geographic DNA data for example, we can better influence the system to accurately predict or recommend tests or exams to doctors.

### C. Machine Learning Module

As noted above, the machine learning module is typically realized in software, i.e., executable instructions stored in the memory of server 230 and executed by the processor. The topology shown in FIG. 2 is illustrative only; the machine learning module may, for example, be implemented in a cloud configuration and deployed on a remote server, receiving input (e.g., feature vectors) from sensor array 210, mobile device 220, server 230 and/or interface device 240.

The machine learning module may implement supervised learning (parametric/non-parametric algorithms, support vector machines, kernels, neural networks), unsupervised learning (clustering, dimensionality reduction, recommender systems, deep learning), or combinations thereof depending on the signals analyzed and the nature of the biomarker. Multiple time-varying signals are well-suited to analysis and classification by a neural network.

Conventional computer programs use an algorithmic approach to problem-solving, i.e., the computer follows a set of instructions in order to solve the problem. Unless the specific steps that the computer needs to follow are known, the computer cannot solve the problem. That restricts the problem-solving capability of conventional computers to problems that we already understand and know how to solve. Biomarkers, however, may not be amenable to algorithmic processing, i.e., the relationship between a time-varying signal and a physiological condition may be complex and unpredictable.

Neural networks process information in a manner similar to the human brain. The network is composed of a large number of highly interconnected processing elements (neurons) working in parallel to solve a specific problem. Neural networks learn by example; they cannot be programmed to perform a specific task. The examples must be selected carefully, otherwise useful time is wasted or, worse, the network might function incorrectly.

Neural networks can recognize diseases using sensor data since there is no need to provide a specific algorithm to identify the disease. Neural networks learn by example, so the details of how to recognize the disease are not needed. What is needed, instead, is a set of examples that are representatives of all the variations of the disease. The examples need to be selected very carefully if the system is to perform reliably and efficiently. Neural networks are particularly well-suited to providing sensor fusion (i.e., combining signal values from several different sensors). Sensor fusion enables a neural network to learn complex relationships among the individual sensor values, which would otherwise be lost if the values were individually analyzed. In medical modeling and diagnosis, this implies that even though each sensor in a set may be sensitive only to a specific physiological variable, a neural network is capable of detecting complex medical conditions by fusing the data from the individual sensors.

Caffe, CUDA, PyTorch, Theano and TensorFlow are suitable neural network platforms (and may be cloud-based or local to an implemented system in accordance with design preferences). The key in realizing the benefits of the invention is to finely tune the neural network to vibro-acoustic and bio-electric signals. In some embodiments, input data includes not only sensor data but portions of the patient's longitudinal health record, which has significant information about the patient's current disease states, medications and medical history.

The input to a neural network may be a vector of input values (or “feature” vector). At least the vibro-acoustic and bio-electric sensors will typically provide output in the form of a time-varying signal, digitized as a sequence of amplitude values. Hence, the neural network (or other machine-learning construct) used herein should be configured to process a plurality of signals, some of which are time-varying signals, as input. This can be accomplished in various ways. One approach to processing time-varying signals is to use a recurrent neural network, in which connections between processing elements form a directed cycle and exhibit dynamic temporal behavior. This facilitates direct analysis of time-varying signals. Another approach, as noted above and which can be implemented on a conventional feedforward (e.g., convolutional or recursive) neural network, repeatedly sample the sensors’ outputs over a synchronized time window. The synchronized raw signal amplitude patterns from each sensor may be combined (e.g., by simple concatenation) into a single feature vector that is used to query the machine learning module, which has previously been trained on similar feature vectors. The time-varying sensor signals may also be processed rather than used in raw form. For example, the short-time Fourier transform may be used to determine the sinusoidal frequency and phase content of discrete portions of a time-varying signal within a time window. In some circumstances, the frequency distribution may provide a more robust feature vector than the amplitude sequence. The frequency distributions of the different signals may be catenated or added together, e.g., with different weights assigned to spectra corresponding to the different signals in order to optimize performance of the neural network.

Processing multiple input parameters—e.g., in addition to the time-varying sensor signals, the input vector may include diverse information such as elements of the patient's health records, the patient's current position and orientation, etc.—can also be accomplished in various ways. As explained above, these different forms of data can be concatenated into a large feature vector, added (e.g., in a weighted fashion), or simply provided as separate inputs to a neural network configured for input fusion.

It should also be noted that neural networks tend to perform better at classification tasks than regression tasks. Hence, if the desired output is a probability (e.g., of the presence of a disease condition), a probability range of 0 to 99 can be divided into sub-ranges (e.g., class probabilities representing each of 10 separate sub-ranges (classes) 0-9, 10-19, 20-29, etc.). If the various input data elements are correlated, an ensemble learning approach can be used. See, e.g., Guo et al., “Input Partitioning Based on Correlation for Neural Network Learning, J. Clean Energy Tech. 1(4):335-38 (2013).

Therefore the neural network will further benefit from various implementations of optimization methods and filters including but not limited to low-pass (LP) filters, high-pass (HP) filters, bandpass (BP) filters, bandstop (BS) filters, infinite-impulse response (IIR) filters and various binary successive approximation (BSA), frequency-response-masking (FRM)-based linear-phase finite-impulse response (FIR) digital filters, and combinations thereof to identify and remove non-physiological signals captured by vibro-acoustic sensors as background “ambient noise” and enhance low threshold sounds.

### D. Applications

As noted, the present invention may be deployed across diverse applications in medicine. Below, we focus on several representative applications.

D.1 Cardiopulmonary Applications

In certain embodiments, the vibro-acoustic, bio-electric, and any number of additional sensors are placed in an array encompassing (or wrapping around) the torso to allow for simultaneous auscultation. Cardiac auscultation can then be simultaneously completed at all four major sites: mitral area (at the apex beat, as the left ventricle is closest to the thoracic cage), tricuspid area (inferior right sternal margin at the point closest to the valve in which auscultation is possible), the pulmonary area (left second intercostal space close to the sternum where the infundibulum is closest to the thoracic cage), and aortic area (right second intercostal space close to the sternum where the ascending aorta is nearest the thoracic cage). Certain sounds such as the aortic and pulmonic sounds are detected best during the S2 heart sound produced by the closing of the semilunar valves of the heart compared to during the S1 heart sound produced by the closing of the atrioventricular valves. Furthermore, according to the disease state and physiological variation from patient to patient, the sounds may be more prominent in certain positions (e.g. sitting up or leaning forward at 45° elicits changes in the amplitude and frequency of mitral valve murmurs as the patient leans forward to move the beating heart wall closer to the chest wall). Similarly, pulmonary auscultation is commonly completed over each of the five lobes of the lungs from both the anterior and posterior sides. With a wrap-around array configuration, more than two, or all cardiac, pulmonary and any additional auscultation sites may be monitored simultaneously, thereby mitigating variations in a patient's breath, position, and condition as can be the case during a traditional auscultation exam. The vibro-acoustic sensors may detect different states of disease in the lungs such as wheezing from asthma, fluid collecting in the base of the lungs that sounds like crackling as the alveolar sacks expand, or pulmonary infections such as pneumonia.

Various wrap-around array configurations may be selected for individual patient variation (e.g. size, body style, gender), duration of use and/or placement, or may be universally adaptable with built-in adjustability for improved data acquisition quality and to be more cost-effective. For example, the use may dictate the type of adhesive option selected for the sensor array from: 1) no adhesive for use in garments, 2) adhesive for sensitive skin that can be removed and re-applied multiple times, 3) sports-grade adhesive that will last, e.g., 15 days, and 4) veterinary-grade adhesive for livestock. As a cost-savings example, certain components such as the wireless electronics module may be reusable whereas the sensor array and adhesives may be disposable. In the flexible sensory array embodiments, the flexible portions may further include strain gauges (e.g., MEMS-based) to additionally record stretching and movement of the localized skin under the patch.

For example, the sensor array may have a substantially straight-line configuration with flexible curvature to align with the contour of various portions of the body, or may have a curved (e.g., U or C shape) configuration enabling one or more sensors to be conveniently positioned over each of the patient's auscultation points. Alternatively, the sensor array may take the form of a wearable vest with an array of connected sensors arranged to monitor torso organs and detect adventitious breath sounds, which are abnormal sounds that are heard over a patient's lungs and airways. These sounds include abnormal sounds such as fine and coarse crackles (sometimes called rales), wheezes (sometimes called rhonchi), pleural rubs and stridor. Adventitious breath sounds are important signs used for diagnosing numerous cardiac and pulmonary conditions. The sensor array signals may thereby be translated into respiration rate, breathing pattern, and posture data.

In another embodiment, using bio-electric sensors and vibro-acoustic sensors combined with machine learning, in addition to the system recording and identifying the P wave, QRS complex, T waves, and U waves, systems in accordance herewith may identify and track digital biomarkers based on H-wave peaks corresponding to the timing of the His bundle depolarization, a feature not normally observed in conventional surface ECGs (0.5-30 Hz bandwidth). When combined with vibro-acoustic sensors, the effect of this H wave peak on the cardiac output, valve murmurs and carotid artery flow, when time-synchronized, can determine diseased properties of the heart's biology such as the sources of arrhythmias, the effect of the arrhythmia on the heart, locations of myocardial infarction or worsening of a clinically significant valve murmurs. The time relation between the H peak and the atrial and ventricular depolarizations in the heart is a useful diagnostic signature that conventionally can only be monitored using invasive intracardiac techniques where the sensor is inserted into an artery via a cardiac catheter.

A complex input system of digital biomarkers may also be combined with environmental inputs to monitor patients with heart failure. After an initial physical evaluation by the physician or clinician, sensors may be placed on the patient's torso (e.g., integrated within a turtleneck garment, shirt, vest or jacket) to maximize the signal recording and establish a baseline for the patient's auscultation sounds, heart rate, bowel sounds, and/or electrical activity during physical maneuvers (tracked by the position sensor) and other non-invasive monitoring inputs. A patient diagnosed with heart failure may be fitted with a sensor array (e.g., in the form of a horseshoe) to monitor cardiopulmonary signals during the subsequent 30 days. During this follow-up time period, sensed environmental conditions (e.g., from a smart scale (providing weight loss/gain and impedance (fat gain/loss) data), a smart toilet's notice of urine color change (indicating hydration status), and/or a smart car (showing decrease in reaction time indicating mental and/or physiological status)), may be combined with other system sensors measuring, for example, increased fluid in the lungs (crackling at the base of the lungs indicates fluid buildup as picked up by the vibro-acoustic sensor), dyspnea (shortness of breath after climbing up stairs as measured by detection of labored breathing and the sensed position of the patient on those stairs) and distension in the carotid artery as picked up by the vibro-acoustic sensor and position sensors over the base of the neck. It should be noted that the bio-electric sensors may not detect any pathology or changes in the ECG, but may nonetheless serve as a reference correlating the opening and closing of each valve in relation to sensed fluid flows. In this example, no single sensor can diagnose heart failure, but a collection of evaluated signals may provide a high degree of statistical confidence that a particular patient has early or late stage heart failure. Furthermore, patterns of the onset of this activity across populations and wide demographics of patients, when correlated with their DNA for personalized medicine, can enable prediction of the onset of disease, and give the care team the option to adjust medications or escalate care. After enough training, systems in accordance herewith may be capable of intervening autonomously or at least suggesting changes to the patient's medication and treatment regimen.

Clinicians and nurses delivering babies can be an overwhelming experience for the clinician and the mother, so having a hands-free system whereby the above embodiments and combinations thereof can further be incorporated into an automated voice-command feedback system allows clinician to obtain and record data quickly, thereby reducing procedure time dramatically and allowing the clinician to focus. In addition, when clinicians by using the same equipment and process, variability among clinicians' assessments and diagnoses can be reduced or at least correlated as well as creating multiple reproducible data points per individual patient so that a baseline normal state can be created and any disease progression (either recovering or worsening of condition) can be tracked.

Additionally, all of the above applications benefit from further physiological response data obtained from the optional sensors described or from a database of recorded environmental data at the relevant location.

D.2 Vascular Surgery Application

When the wall of a blood vessel weakens, a balloon-like dilation called an aneurysm sometimes develops. This happens most often in the abdominal aorta, an essential blood vessel that supplies blood to the legs. Every year, 200,000 people in the U.S. are diagnosed with an abdominal aortic aneurysm (AAA). The most common treatment is the placement of an aortic abdominal graft through endovascular surgery in which a synthetic graft is inserted through the femoral artery and threaded up to the aorta with a catheter. The graft is placed at the site of the aneurysm and reinforces the weakened section of the aorta to prevent rupture.

If an aortic abdominal graft ruptures, the patient will quickly lose so much blood s/he may die. There is currently no commonly accepted way to tell if the graft is failing. A wearable or implantable vibro-acoustic sensor may be used—e.g., in conjunction with a CPU and a neuromorphic processor along with memory and communications capability—to detect graft failure. The sensor array may be worn externally or implanted next to a just-completed aortic abdominal graft. An embedded neuromorphic processor is trained on the sounds of blood flowing past the just-introduced graft. This training phase occurs over a relatively short period of time, e.g., a few days, following which the neuromorphic processor is switched into diagnostic mode. The wearable or implant then communicates (e.g., wirelessly) with an external store-and-forward device that relays information to a call center and/or prescribing clinician, or stores data for proximate or remote retrieval. More generally, various embodiments of the sensor array described herein may be implantable and placed near a surgical site to monitor recovery and detect the need for follow-up treatment.

As noted, the present invention may be deployed across diverse applications in medicine. Other medical applications include but are not limited to: anesthesiology, dermatology, endocrinology, gastroentology, hematology, ophthalmology, pathology, radiology, urology, professional sports medicine, physical therapy, etc. The sensor applications may uncover previously unknown correlations among the various fields. The sensor array may alternatively be used for personal health and fitness.

D.3 Non-Human Applications

Bovine respiratory disease (“BRD”) is the most common disease affecting cattle in North America. BRD affects the respiratory tracts and can often be fatal, causing billions of dollars in economic losses for ranchers, dairymen and feed lot operators. Just as in humans, digital biomarkers of BRD may be created. Using sensor arrays as described herein, producers (e.g. ranchers, dairymen, feed lots and veterinarians) can detect BRD early, determine the severity of the disease and select an appropriate treatment regimen, which may help them improve cardiopulmonary-health related outcomes.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. A system for receiving and transducing biological events into electrical signals and diagnosing a medical condition based thereon, the system comprising:
a sensor array comprising a vibro-acoustic sensor for measuring body sounds of a target living organism and a bio-electric sensor for measuring a bio-electric signal of the target living organism;
a processor; and
a machine learning module, executable by the processor and trained on signals characteristic of the sensor array, the machine learning module receiving signals from the sensors and, based on the training, outputting a probability indicative of a physiological condition.

2. The system of claim 1, wherein the physiological condition is a biomarker.

3. The system of claim 1, wherein the sensor array further comprises one or more sensors for measuring at least one environmental stimulus or condition.

4. The system of claim 3, wherein the at least one environmental stimulus or condition is at least one of skin temperature, ambient temperature, barometric pressure, 9-axis motion geolocation, location-dependent real-time weather conditions, galvanic skin response, or pollution.

5. The system of claim 1, wherein the sensor array comprises at least one sensor for measuring at least one of wavelength transmittance/absorbance, oxygen saturation, ambient skin temperature, core temperature, ACG, BCG, VCG, EKG, EMG, EOG, EEG, VOC excretion or vocal tonal inflection.

6. The system of claim 1, wherein the sensor array further comprises an optical sensor.

7. The system of claim 1, further comprising a database of longitudinal health records, the health record of a target living organism being monitored by the sensors providing an input to the machine learning module.

8. The system of claim 1, wherein the machine learning module is a neural network.

9. The system of claim 8, wherein the neural network is a recurrent neural network.

10. The system of claim 8, wherein the neural network is a feedforward neural network.

11. The system of claim 8, wherein the neural network is an ensemble of neural networks.

12. The system of claim 1, wherein the vibro-acoustic sensor and the bio-electric sensor each produce time-varying signals, the signals received by the machine learning module from the vibro-acoustic sensor and the bio-electric sensor being time-synchronized.

13. The system of claim 12, wherein the signals are received by the machine learning module as catenated raw amplitude sequences.

14. The system of claim 12, wherein the signals are received by the machine learning module as combined short-time Fourier transform spectra.

15. The system of claim 1, wherein the sensor array is connected by wires.

16. The system of claim 1, wherein the sensor array communicates wirelessly.

17. The system of claim 1, wherein the machine learning module is remote from the sensors and in communication therewith via a network.

18. The system of claim 1, further comprising at least one acoustic stimulus generators.
